(19) United States
(12) Patent Application Publication
(10) Pub. No.: US 2026/0346025 A1
(43) Pub. Date: Nov. 5, 2026

(54) SYSTEM AND METHOD FOR TRUNCATING PHYSIOLOGICAL DATA

(71) Applicant: Medtronic, Inc., Minneapolis, MN (US)

(72) Inventors: Kristin N. Hageman, Dayton, MN (US); Joshua J. Nedrud, Roseville, MN (US); Joel A. Anderson, Brooklyn Park, MN (US); Thaddeus S. Brink, St. Paul, MN (US); Philip E. Tracton, Porter Ranch, CA (US)

(21) Appl. No.: 19/659,439

(22) Filed: Apr. 27, 2026

Related U.S. Application Data
(60) Provisional application No. 63/799,044, filed on May 2, 2025.

(51) Int. Cl. A61N 1/365

(52) U.S. Cl. CPC A61N 1/365

(57) ABSTRACT

An example implantable medical device includes sensing circuitry configured to: sense a physiological signal of a patient; and generate, based on the physiological signal, information comprising a plurality of sample points that are representative of the physiological signal; a memory configured to store a machine learning (ML) model; and processing circuitry configured to truncate the information based on saliency values for at least some sample points of the plurality of sample points; and apply the truncated information to the ML model to determine a presence of an evoked compound action potential (ECAP) in the sensed physiological signal.

### CROSS-REFERENCE TO RELATED APPLICATION

This application claims the benefit of and priority to U.S. Provisional Patent Application No. 63/799,044 filed May 2, 2025, the entire disclosure of which is incorporated by reference herein.

### TECHNICAL FIELD

The disclosure relates generally to medical device systems and, more particularly, medical device systems configured to detect physiological signals.

### BACKGROUND

Medical devices may be used to monitor physiological signals of a patient. For example, some medical devices are configured to sense cardiac electrogram (EGM) signals indicative of the electrical activity of the heart via electrodes, electroencephalogram (EEG) signals indicative of the electrical activity of the heart via electrodes, and/or an evoked compound action potential (ECAP). Some medical devices may be configured to deliver a therapy in conjunction with or separate from the monitoring of physiological signals.

### SUMMARY

A medical device, such as an implantable medical device (IMD), may be configured to sense one or more physiological signals of a patent. Example physiological signals may include at least one of an evoked compound action potential (ECAP), electroencephalogram (EEG) signal, electrocardiogram (ECG) signal, respiration, blood pressure, activity level, electromyography (EMG) signal, evoked potential, intrinsic nerve activity, and/or electrophysiology (EP). These signals may be sensed using one or more sensors, such as one or more comprise electrodes, accelerometers, or any other known sensors.

In accordance with techniques described herein, a medical device, such as an IMD, may truncate information representative of sensed physiological signal(s) (e.g., a continuously sensed signal or a number of sensed signals at discrete times over a period of time) and apply the truncated information to a machine learning (ML) model. The IMD may be configured to truncate the information representative of sensed physiological signal(s) based on saliency values of sample points of the information representative of sensed physiological signal(s). The ML model may be a convolutional neural network in some examples. The IMD may be configured to apply the truncated information to the ML model to determine whether an ECAP is detected in the sensed physiological signal. In this manner, the ML model may be configured to generate output for the ML model using the truncated information that can save computational resources and/or improve efficiency of running the ML model.

In some examples, the techniques described herein may increase accuracy or at least maintain sufficient accuracy of the output of the ML model (e.g., whether an ECAP is detected in the sensed physiological signal) while improving efficiency of using computational resources, such as memory or power, while running a ML model. Applying smaller data sets to the ML model may reduce computations compared to larger data sets. For example, an IMD truncating sensed physiological data, in accordance with techniques described herein, that is configured to operate with improved computational efficiency may enable the IMD to have a greater lifespan and/or enable the IMD to run the ML model faster or more often, which may help produce improved clinical results for a patient.

In one example, this disclosure describes an implantable medical device comprising: sensing circuitry configured to: sense a physiological signal of a patient; and generate, based on the physiological signal, information comprising a plurality of sample points that are representative of the physiological signal; a memory configured to store a machine learning (ML) model; and processing circuitry configured to: truncate the information based on saliency values for at least some sample points of the plurality of sample points; and apply the truncated information to the ML model to determine a presence of an evoked compound action potential (ECAP) in the sensed physiological signal.

In another example, this disclosure describes a method comprising: sensing, by sensing circuitry, a physiological signal of a patient; generating, by the sensing circuitry and based on the physiological signal, information comprising a plurality of sample points that are representative of the physiological signal; truncating, by processing circuitry, the information based on saliency values of at least some sample points of the plurality of sample points; and applying, by the processing circuitry, the truncated information to a machine learning (ML) model to determine a presence of an evoked compound action potential (ECAP) is detected in the physiological signal.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1A illustrates the environment of an example medical system in conjunction with a patient, in accordance with one or more techniques disclosed herein.

FIG. 1B illustrates the environment of an example medical system in conjunction with a patient, in accordance with one or more techniques disclosed herein.

FIG. 2 is a functional block diagram illustrating an example configuration of the implantable medical device (IMD) of the medical system of FIG. 1A or FIG. 1B in accordance with one or more techniques disclosed herein.

FIG. 3 is a graph of an example evoked compound action potentials (ECAPs) sensed for respective stimulation pulses.

FIG. 4 is a conceptual diagram illustrating an example technique for truncating a sensed physiological signal, in accordance with one or more techniques disclosed herein.

FIG. 5A is a conceptual diagram illustrating an example of generating a linear transformation, in accordance with one or more techniques disclosed herein.

FIG. 5B is a conceptual diagram illustrating an example of truncating physiological data, in accordance with one or more techniques disclosed herein.

FIG. 6 is a functional block diagram illustrating an example configuration of the external device of FIGS. 1A-1B, in accordance with one or more techniques disclosed herein.

FIG. 7 is a block diagram illustrating an example system that includes an access point, a network, external computing devices, such as a server, and one or more other computing devices, which may be coupled to the IMD and external device of FIGS. 1-5, in accordance with one or more techniques disclosed herein.

FIG. 8 is a flow diagram illustrating an example technique for applying a physiological signal to a machine learning model to generate an output, in accordance with one or more techniques disclosed herein.

Like reference characters denote like elements throughout the description and figures.

### DETAILED DESCRIPTION

A variety of types of implantable medical devices (IMDs) sense physiological signals that include physiological data, such as ECAPs, cardiac EGMs, or EEGs. In some examples, EGMs may also include electrocardiogram (ECGs or EKGs). In some examples, electrodes used by IMDs to sense physiological signals are typically integrated with a housing of the IMD and/or coupled to the IMD via one or more elongated leads. Example IMDs that monitor physiological data and/or provide therapy include spinal cord stimulators, deep brain stimulation devices, and pacemakers and implantable cardioverter-defibrillators. These devices may be coupled to intravascular and/or extravascular leads, as well as include with housings configured for implantation within the heart or other anatomical location that are leadless (e.g., carry electrodes on the housing). An example of pacemaker configured for intracardiac implantation is the Micra™ Transcatheter Pacing System, available from Medtronic plc. Some IMDs that do not provide therapy, e.g., implantable patient monitors, sense cardiac EGMs. Examples of such an IMD are the Reveal LINQ™ and LINQ II™ Insertable Cardiac Monitors, available from Medtronic plc, which may be inserted subcutaneously. Such IMDs may facilitate relatively longer-term monitoring of patients during normal daily activities and may periodically transmit collected data to a network service, such as the Medtronic Carelink™ Network.

In some examples, an IMD may sense a particular signal may also sense, or determine, an amount of noise. On some occasions, the amount of noise included in the sensed signal may make the desired sensed signal, such as an ECAP, EEG signal, or ECG signal, more difficult to identify in a signal sensed by the IMD. In some examples, an IMD may input data representative of the one or more of the sensed physiological signals into a machine learning (ML) model to into a ML model to generate an output, as a determination and/or prediction one or more physiological parameters, diagnostic parameters, and/or therapeutic parameters.

An IMD may have limited battery and/or limited memory size for an IMD to be small enough to be effectively implantable in a patient. The power and/or memory needs for applying most or all sample points of a physiological signal to a ML model are often high, which may be problematic for an IMD due to the limited battery and/or the limited memory size of an IMD. Furthermore, the processing power of an IMD may be limited compared to high end workstations or servers. Therefore, an IMD may not be able to run all sensed data quickly enough through an ML model in order to adjust therapy using the output of the ML model.

In accordance with techniques described herein, a medical device, such as an IMD, may truncate information representative of sensed physiological signal(s) (e.g., a continuously sensed signal or a number of sensed signals at discrete times over a period of time) and apply the truncated information to a machine learning (ML) model. The IMD may truncate the information by removing one or more sample points of a plurality of sample points representative of the sensed physiological signals. In some examples, the IMD may be configured to truncate the sensed information based on saliency values of sample points of the information representative of a sensed physiological signal.

In some examples, the IMD may be configured to apply the truncated information to the ML model to generate an output, such as a determination of whether an ECAP is detected in the sensed physiological signal (e.g., determination that the ECAP is present in the sensed physiological signal or the ECAP is not present). In some examples, the ML model may output a characteristic of the ECAP (e.g., a magnitude of the ECAP) that may characterize the ECAP and/or indicate whether or not the ECAP is present in the sensed physiological signal. In this manner, the ML model may be configured to generate output for the ML model using the truncated information that can save computer resources, improve efficiency, and/or improve the speed, of running the ML model. In some examples, the techniques described herein may increase accuracy or at least maintain sufficient accuracy of the output of the ML model (e.g., whether an ECAP is detected in the sensed physiological signal) while improving efficiency of using computer resources while running a ML model. Computer resources of the IMD may include battery power or memory.

An IMD may be repeatedly performing such continuous measurements of physiological signals over long periods of time (e.g., days, months, or years). Truncating information representative of sensed physiological data, such as based on saliency values of sample points of the sensed physiological data, to more efficiently use computer resources of the IMD, while mainlining or improving accuracy of the output of a ML model, may enable an IMD to have a longer implantable life and/or may enable greater use of a ML model in an IMD. An IMD being able to generate truncated physiological data to more efficiently run a ML model more often may enable more accurate health event detections and/or predictions, which may enable improved patient diagnostics and/or improved patient treatment.

Any medical device configured to sense physiological data via implanted electrodes or sensors, including the examples identified herein, may implement the techniques of this disclosure for applying information representative of physiological signal to a ML model to generate an output based on the physiological signal.

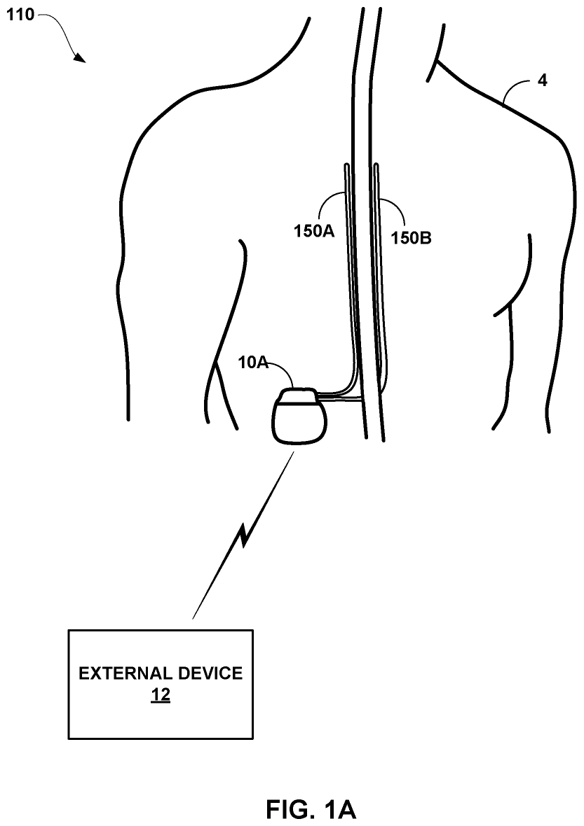

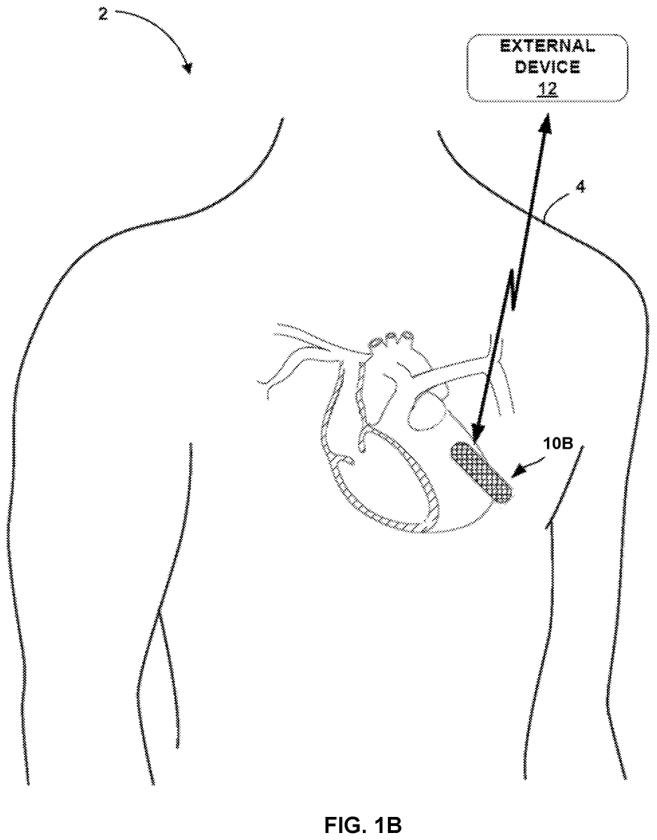

FIG. 1A illustrates a conceptual diagram illustrating an example system 110 that includes another example of an IMD 10A that is configured to deliver spinal cord stimulation (SCS) therapy and an external device 12, in accordance with one or more techniques of this disclosure. Although the techniques described in this disclosure are generally applicable to a variety of IMDs, application of such techniques to IMDs and, more particularly, an ICM or implantable electrical stimulators (e.g., neurostimulators) will be described for purposes of illustration. More particularly, the disclosure will refer to an IMD 10A system or implantable SCS system for purposes of illustration, but without limitation as to other types of medical devices or other therapeutic applications of medical devices.

As shown in FIG. 1A, system 110 includes an IMD 10A, leads 150A and 150B, and external programmer 150 shown in conjunction with a patient 4, who is ordinarily a human patient. In the example of FIG. 1, IMD 10A is an implantable electrical stimulator that is configured to generate and deliver electrical stimulation therapy to patient 4 via one or more electrodes of electrodes of leads 150A and/or 150B (collectively, “leads 150”), e.g., for relief of chronic pain or other symptoms. In other examples, IMD 10A may be coupled to a single lead carrying multiple electrodes or more than two leads each carrying multiple electrodes. In some examples, the stimulation signals, or pulses, may be configured to elicit detectable ECAP signals that are sensed by IMD 10A. In some examples, IMD 10A may use sensed ECAP signals to determine a posture state occupied by patient 4 and/or determine how to adjust one or more parameters that define stimulation therapy. In this manner, IMD 10A may sense one or more physiological signals and use one or more characteristics of those signals as feedback for closed-loop control of the electrical stimulation that is delivered. IMD 10A may be a chronic electrical stimulator that remains implanted within patient 4 for weeks, months, or even years. In other examples, IMD 10A may be a temporary, or trial, stimulator used to screen or evaluate the efficacy of electrical stimulation for chronic therapy. In one example, IMD 10A is implanted within patient 4, while in another example, IMD 10A is an external device coupled to percutaneously implanted leads. In some examples, IMD 10A uses one or more leads, while in other examples, IMD 10A is leadless.

FIG. 1B illustrates the environment of another example medical system 2 in conjunction with a patient 4, in accordance with one or more techniques of this disclosure. The example techniques may be used with an IMD 10B, which may be in wireless communication with at least one of external device 12 and other devices not pictured in FIG. 1. In some examples, IMD 10B is implanted outside of a thoracic cavity of patient 4 (e.g., subcutaneously in the pectoral location illustrated in FIG. 1). IMD 10B may be positioned near the sternum near or just below the level of the heart of patient 4, e.g., at least partially within the cardiac silhouette. IMD 10B includes a plurality of electrodes (not shown in FIG. 1) and is configured to sense a cardiac EGM via the plurality of electrodes. In some examples, IMD 10A takes the form of the Reveal LINQ™ or LINQ II™ ICM, or another ICM similar to, e.g., a version or modification of, the Reveal LINQ™ or LINQ II™ ICMS.

External device 12 may be a computing device with a display viewable by the user and an interface for providing input to external device 12 (i.e., a user input mechanism). In some examples, external device 12 may be a notebook computer, tablet computer, workstation, one or more servers, cellular phone, personal digital assistant, or another computing device that may run an application that enables the computing device to interact with IMD 10A or IMD 10B. External device 12 is configured to communicate with IMD 10A or IMD 10B and, optionally, another computing device (not illustrated in FIG. 1), via wireless communication. External device 12, for example, may communicate via near-field communication technologies (e.g., inductive coupling, NFC or other communication technologies operable at ranges less than 10-20 cm) and far-field communication technologies (e.g., RF telemetry according to the 802.11 or Bluetooth® specification sets, or other communication technologies operable at ranges greater than near-field communication technologies).

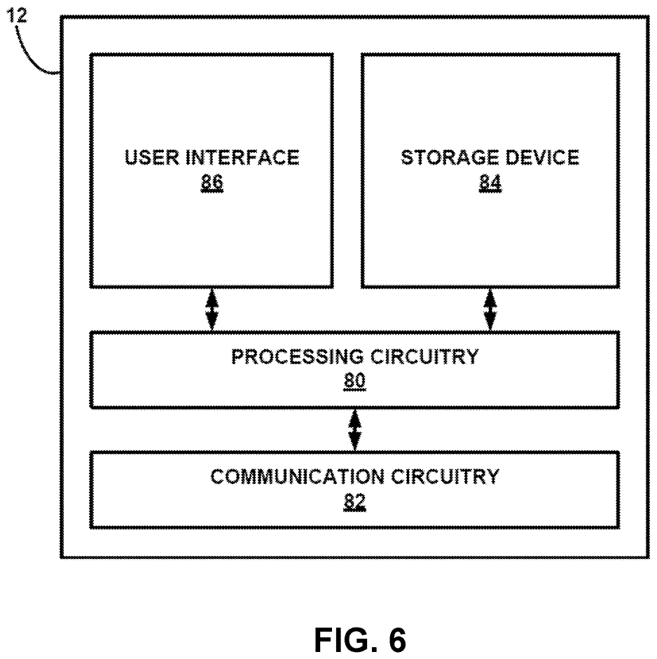

External device 12 may be used to configure operational parameters for IMD 10A or IMD 10B. External device 12 may be used to retrieve data from IMD 10A or IMD 10B. The retrieved data may include values of physiological parameters measured by IMD 10A or IMD 10B, indications of patient health episodes or other maladies detected by IMD 10A or IMD 10B, and physiological signals recorded by IMD 10A or IMD 10B. For example, external device 12 may retrieve physiological data recorded by IMD 10A or IMD 10B. As discussed in greater detail below with respect to FIG. 6, one or more remote computing devices may interact with IMD 10A or 10B in a manner similar to external device 12, e.g., to program IMD 10A or 10B and/or retrieve data from IMD 10A or 10B, via a network.

Processing circuitry of medical system 2, e.g., of IMD 10A, IMD 10B, external device 12, and/or of one or more other computing devices, may be configured to perform the example techniques of this disclosure for truncating information representative of sensed physiological signal(s) and applying the truncated information to a ML model to generate output, such as determining whether an ECAP is detected in the sensed physiological signal(s).

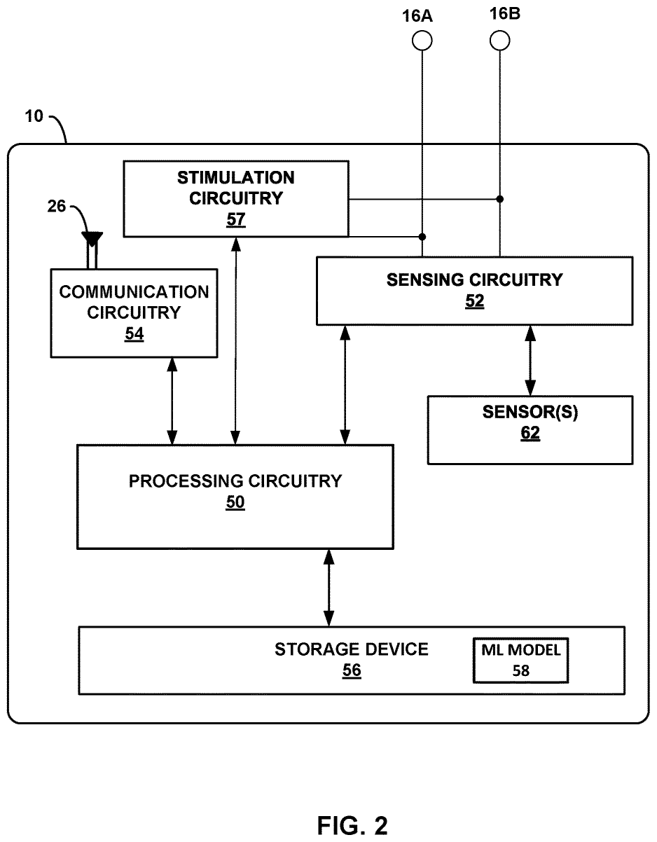

FIG. 2 is a functional block diagram illustrating an example configuration of IMD 10, which is example of IMD 10A of FIG. 1A or IMD 10B of FIG. 1B in accordance with one or more techniques described herein. In the illustrated example, IMD 10 includes electrodes 16A and 16B (collectively “electrodes 16”), antenna 26, processing circuitry 50, sensing circuitry 52, communication circuitry 54, storage device 56, stimulation circuitry 57, and sensors 62. Although the illustrated example includes two electrodes 16, IMDs including or coupled to more than two electrodes 16 may implement the techniques of this disclosure in some examples. However, IMD 10 does not need to include all these components in some examples, or IMD 10 may include additional components in some examples. For example, IMD 10 may not include stimulation circuitry 57, in some examples.

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may sense signals from electrodes 16 or sensors 62, e.g., to produce an ECAP signal, cardiac EGM, EEG signal, optical signal ECAP signal, or other signal in order to facilitate monitoring one or more particular physiological signals of the patient. Stimulation circuitry 57 may be coupled to electrodes 16 and configured to cause electrical stimulation therapy to be delivered to patient 4 via one or more electrodes 16. Sensing circuitry 52 also may monitor signals from sensors 62, which may include one or more accelerometers, pressure sensors, and/or optical sensors, as examples. In some examples, electrodes 16 may be referred to as sensors, such as when being used to detect a physiological signal, such as an ECAP signal, cardiac EGM signal or EEG signal. In some examples, sensing circuitry 52 may include one or more filters and amplifiers for filtering and amplifying signals received from electrodes 16 and/or sensors 62.

Sensing circuitry 52 may also provide one or more digitized physiological signals to processing circuitry 50 for analysis, e.g., to apply information representative of the sensed physiological signal to a ML model 58 to generate an output with respect to the sensed physiological signal 130, such as a determination a physiological parameter and/or health condition of the patient according to the techniques of this disclosure. Sensing circuitry 52 may sense the physiological signals in the form of a voltage or potential difference between electrodes. Sensing circuitry 52 can then generate information representing the sensed physiological signals, which may include a plurality of sample points. These sample points may be analog or digital values representing a respective portion of the sensed physiological signals. In some examples, processing circuitry 50 may store the digitized physiological signals in storage device 56.

Communication circuitry 54 may include any suitable hardware, firmware, software or any combination thereof for communicating with another device, such as external device 12, another networked computing device, or another IMD or sensor. Under the control of processing circuitry 50, communication circuitry 54 may receive downlink telemetry from, as well as send uplink telemetry to external device 12 or another device with the aid of an internal or external antenna, e.g., antenna 26. In addition, processing circuitry 50 may communicate with a networked computing device via an external device (e.g., external device 12) and a computer network, such as the Medtronic CareLink® Network. Antenna 26 and communication circuitry 54 may be configured to transmit and/or receive signals via inductive coupling, electromagnetic coupling, Near Field Communication (NFC), Radio Frequency (RF) communication, Bluetooth, WiFi, or other proprietary or non-proprietary wireless communication schemes.

In some examples, storage device 56 includes computer-readable instructions that, when executed by processing circuitry 50, cause IMD 10 and processing circuitry 50 to perform various functions attributed to IMD 10 and processing circuitry 50 herein. Storage device 56 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. In some examples, storage device 56 may be referred to as a memory. Storage device 56 may store one or more ML models 58, such as described herein. Storage device 56 may store, as examples, programmed values for one or more operational parameters of IMD 10 and/or data collected by IMD 10 for transmission to another device using communication circuitry 54. Data stored by storage device 56 and transmitted by communication circuitry 54 to one or more other devices may include digitized physiological signals, such as digitized cardiac EGMs or digitized EEGs, as examples. In some examples, storage device 56 may refer to some or all of memory located on IMD 10, including memory used by ML model 58.

In some examples, stimulation circuitry 57 is configured to generate and deliver electrical therapy to the patient. Stimulation circuitry 57 may include one or more pulse generators, capacitors, and/or other components capable of generating and/or storing energy to deliver as electrical stimulation therapy.

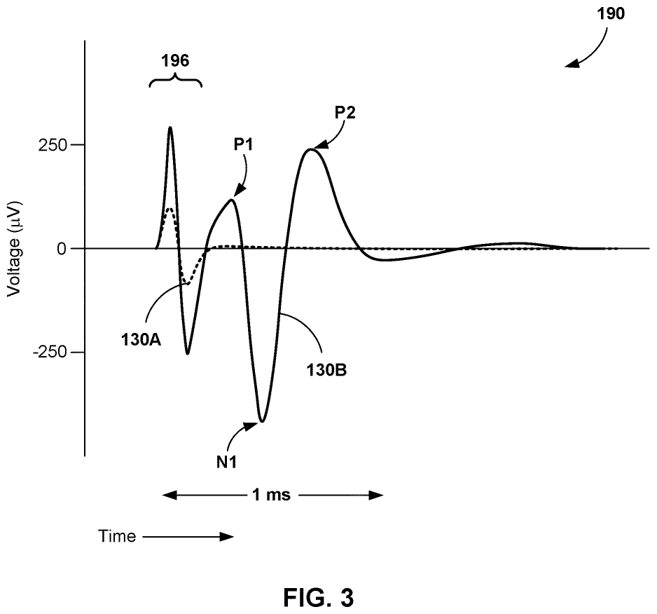

FIG. 3 is a graph 390 of an example evoked compound action potentials (ECAPs) sensed for respective stimulation pulses. As shown in FIG. 3, graph 190 shows example non-ECAP signal 130A (dotted line) and ECAP signal 130B (solid line). Each of signals 130A and 130B may be sensed from control pulses that were delivered from a guarded cathode and bi-phasic pulses including an interphase interval between each positive and negative phase of the pulse. In some examples, a guarded cathode of the stimulation electrodes may be located at the end of an 8-electrode lead while two sensing electrodes are provided at the other end of the 8-electrode lead. Signal 130A illustrates the voltage amplitude sensed as a result from a sub-threshold stimulation pulse. Peaks 196 of non-ECAP signal 130A are detected and represent the artifact of the delivered control pulse. However, no propagating signal is detected after the artifact in signal 130A because the control pulse was sub-threshold. In this manner, no ECAP is present in signal 130A.

In contrast to signal 130A, ECAP signal 130B represents the voltage amplitude detected from a supra-threshold control pulse. Peaks 196 of ECAP signal 130B are detected and represent the artifact of the delivered control pulse. After peaks 196, ECAP signal 130B also includes peaks P1, N1, and P2, which are three typical peaks representative of propagating action potentials from an ECAP (e.g., an ECAP is detectable and present within the signal 130B). While FIG. 3 is not drawn to scale, peaks 196 of ECAP signal that represent the artifact of the delivered control pulse have a higher amplitude than peaks P1, N1, and P2 of the ECAP signal that represent three typical peaks representative of propagating action potentials from an ECAP. In some examples, the amplitude of the artifact peaks 196 and amplitude of points near the artifact peaks 196 of the stimulation pulse itself may obscure the lower amplitudes of the ECAP signal, such as at least one of peaks P1, N1, and P2 that represent propagating action potentials from an ECAP.

The example duration of the artifact and peaks P1, N1, and P2 is approximately 1 millisecond (ms). When detecting the ECAP of ECAP signal 130B, different characteristics may be identified. For example, a characteristic of the ECAP may be the amplitude between N1 and P2. This N1-P2 amplitude may be easily detectable even if the artifact impinges on P1, a relatively large signal, and the N1-P2 amplitude may be minimally affected by electronic drift in the signal. In other examples, a characteristic of the ECAP used to control informed pulses may be an amplitude of P1, N1, or P2 with respect to neutral or zero voltage. In some examples, a characteristic of the ECAP used to control informed pulses may be a sum of two or more of peaks P1, N1, or P2. In other examples, the characteristic of ECAP signal 130B may be the area under one or more of peaks P1, N1, and/or P2. In other examples, a characteristic of the ECAP may be a ratio of one of peaks P1, N1, or P2 to another one of the peaks. In some examples, the characteristic of the ECAP may be a slope between two points in the ECAP signal, such as the slope between N1 and P2. In other examples, the characteristic of the ECAP may be the time between two points of the ECAP, such as the time between N1 and P2. The time between two points in the ECAP signal may be referred to as a latency of the ECAP and may indicate the types of fibers being captured by the control pulse. ECAP signals with lower latency (i.e., smaller latency values) indicate a higher percentage of nerve fibers that have faster propagation of signals, whereas ECAP signals with higher latency (i.e., larger latency values) indicate a higher percentage of nerve fibers that have slower propagation of signals. Other characteristics of the ECAP signal may be used in other examples.

The amplitude of the ECAP signal increases with increased amplitude of the control pulse, as long as the pulse amplitude is greater than threshold such that nerves depolarize and propagate the signal. The target ECAP characteristic (e.g., the target ECAP amplitude) may be determined from the ECAP signal detected from a control pulse when informed pulses are determined to deliver effective therapy to the patient. The ECAP signal thus is representative of the distance between the stimulation electrodes and the nerves appropriate for the stimulation parameter values of the informed pulses delivered at that time. Therefore, IMD 10 may attempt to use detected features of a measured ECAP characteristic value to change informed pulse parameter values and maintain the target ECAP characteristic value during informed pulse delivery.

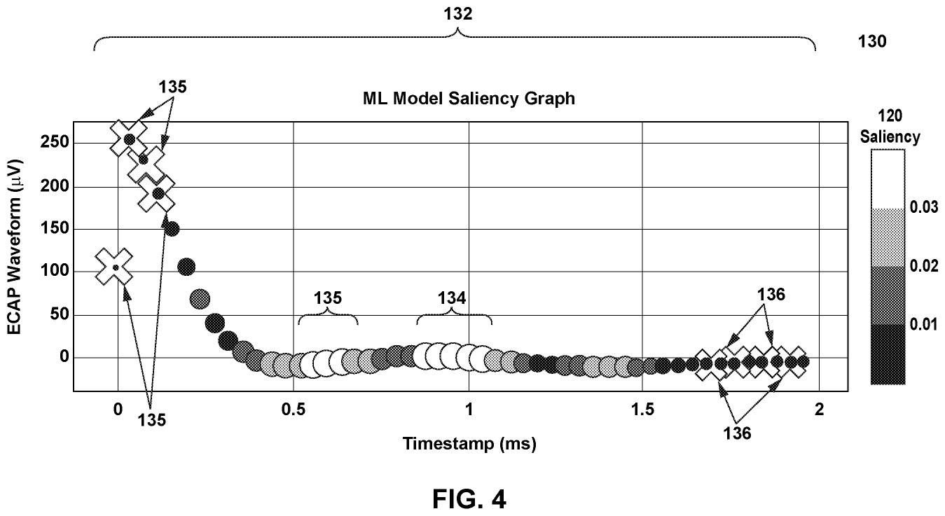

FIG. 4 is a conceptual diagram illustrating an example of non-linearly truncating or decimating information representative of physiological signal 130 based on saliency values 120 of respective sample points of the information representative of physiological signal. In some examples, truncating information representative of physiological signal 130 based on saliency values 120 may be non-linearly truncating the information representative of physiological signal 130 before applying the truncated signal to ML model 58. As shown in the example of FIG. 4, physiological signal 130 is an ECAP signal. In some examples, physiological signal 130 may be other types of physiological signals, such as an ECG signal, an EGM signal, an EEG signal, an EMG signal, or other physiological signal. In some examples, physiological signal 130 may be a raw sensed signal. In some examples, physiological signal 130 may be a preprocessed signal based on a raw sensed signal.

In some examples, as shown in FIG. 4, information representative of physiological signal 130 comprises a plurality of sample points 132. In some examples, one or more of the sample points 132 of the information has a respective saliency value 120. In some examples, a saliency value 120 indicates a relative importance of a particular sample point in respective information representative of physiological signal 130 in determining a respective output of the ML model 58. For example, as shown in FIG. 4, the saliency values 120 for the respective sample points 132 indicate a relative importance of those sample points 132 in determining whether physiological signal 130 is an ECAP signal (e.g., physiological signal 130 is an ECAP signal as physiological signal 130 is a result from a supra-threshold control pulse, such as ECAP signal 130B, as shown in FIG. 3) or is not an ECAP signal (e.g., physiological signal 130 is not an ECAP signal as physiological signal 130 is a result from a sub-threshold stimulation pulse, such as signal 130A, as shown in FIG. 3).

As shown in FIG. 4, sample points 135 of information representative of physiological signal 130 have among the highest amplitude values or highest variability of the sample points 132 of information representative of physiological signal 130. However, sample points having the greatest amplitude value or highest variability does not necessarily correspond to those respective sample points being the most important sample points when applying information representative of a physiological signal to ML model 58 to generate an output. This may be particularly true when trying to identify an ECAP from the data present. Indeed, those high amplitude sample points 135 may be representative of the stimulus itself (e.g., an artifact) instead of the propagating ECAP signal that the system is interested in identifying.

In some examples, saliency values 120 may be determined, such as by ML model 58, based on averaging the absolute values of the backpropagation gradients at a plurality sample points 132 on a plurality of training ECAP signal data. In other examples, the saliency value 120 may be determined based on averaging the squared values of the backpropagation gradients at a plurality sample points 132 on a plurality of training ECAP signal data. In some examples, saliency values may be determined based on using the Shapley equation.

In some examples, ML model 58 may be trained on a plurality of training ECAP signal data and ML model 58 may determine and store saliency values 120 of at least some of the plurality sample points 132 of information representative of physiological signal 130, such as an ECAP signal. In some examples, processing circuitry 50 may be configured to determine the saliency values 120 for the respective sample points 132.

In some examples, a saliency value 120 of a sample point 132 of information representative of physiological signal 130 may correspond to an amount of an effect changing that particular sample point by a value, such as 1, will have on an output of the ML model 58. For example, changing a value of one of sample points 134 by 1 may change a value of the output of ML model 58 by greater than 0.03. In addition, for example, changing a value of one of sample points 135 or 136 may change a value of the output of ML model 58 by less than 0.01 or less than 0.005. An amount or percentage/ratio an output of ML model 58 changes based on changing a value of a particular sample point by a particular amount, such as 1, corresponds to the saliency value 120 of that particular sample point. In some examples, saliency values 120 of a plurality of sample points 132 of information representative of physiological signal 130 may be determined during training of ML model 58 by performing backpropagation saliency on ML model 58 during training of ML model 58.

In some examples, sample points 133, which may be near P1 peak of information representative of physiological signal 130, and sample points 134, which may be near N1 peak of information representative of physiological signal 130, may have the highest saliency values (e.g., are highly important at least compared to other points). In some examples, sample points 135, which may be near the artifact peaks 196 of the information representative of physiological signal 130, and sample points 136, which may be near the end of the information representative of physiological signal 130, may have the lowest saliency values (e.g., are the least important). However, while the saliency values of the sample points may correspond to particular aspects of the ECAP signal, a saliency value of a sample point is determined based on the importance of the particular sample point to the generation of the output of the ML model 58.

In some examples, processing circuitry 50 may truncate information representative of physiological signal based on saliency values 120 for at least some of the plurality of sample 132 by discarding at least some of the sample points 135, 136 that have a saliency value below a saliency value threshold. In some examples, a saliency value threshold may be up to 0.005. In some examples, a saliency value threshold may be up to 0.01. In some examples, a saliency value threshold may be based on an amount or percentage of sample points 132 to be truncated, such as a pre-determined amount or percentage of sample points 132 to be truncated. For example, a saliency value threshold may be based on truncating up to a 75^{th} percentile (or any other determined percentile over zero) of the sample points 132.

In some examples, processing circuitry 50 may truncate information representative of physiological signal by discarding at least some of the sample points, such as artifact sample points 135 and/or late signal sample points 136, of the physiological signal 130 that have the lowest saliency values (e.g., below a saliency value threshold). In some examples, processing circuitry 50 may additionally or alternatively adjust a weighting ML model 58 is to apply to respective sample points 132 of information representative of physiological signal 130 based on the respective saliency values of at least some of the respective sample points 132. In some examples, processing circuitry 50 may adjust a weighting ML model 58 is to apply to respective sample points 132 of truncated information representative of physiological signal 130 based on the respective saliency values of at least some of the respective sample points 132. For example, ML model 58 may be adjusted to weight the sample points with the greatest saliency value (e.g., above a weighting threshold) with greater weight, such was weighing the sample points 133, which may be near P1 peak, and/or sample points 134, which may be near the N1 peak with greater weight. In some examples, processing circuitry 50 may adjust weights of ML model 58 based on the saliency values 120 of respective sample points 132 of information representative of physiological signal 130 and apply the adjusted weights of ML model 58 to the respective samples points of the information representative of physiological signal 130 or the truncated information representative of physiological signal.

In some examples, when sample points of information representative of physiological signal 130 have saliency values that are low that indicate the informational value of those respective sample points are low, processing circuitry 50 may truncate information representative of physiological signal 130 by generating an average value of those respective sample points and truncating the sample points to be represented by less sample points, such as 1. In some examples, processing circuitry 50 may truncate information representative of physiological signal by averaging a value of a plurality of sample points having a saliency value in a particular saliency value window, such as sample points 136, to generate at least one truncated sample point to represent a plurality of sample points 132 of the physiological signal 130. For example, if there are eight sample points having a saliency value corresponding to sample points 136, processing circuitry 50 may generate an average value of those eight sample points and replace the eight sample points with one sample point having the average value (e.g., the eight sample points of information representative of physiological signal are truncated to one sample point in the truncated information representative of physiological signal).

In some examples, processing circuitry 50 may be configured to truncate the information representative of sensed physiological signal 130 and apply the truncated information representative of physiological signal to ML model 58 to generate an output, such as whether an ECAP signal from a supra-threshold control pulse is detected. In some examples, processing circuitry 50 may apply the truncated information representative of physiological signal to ML model 58 to generate an output, such as whether the detected a health condition status or prediction of the patient 4 or a determination of one or more features of the information representative of physiological signal 130 that may be indicative of a health condition status or prediction.

In some examples, processing circuitry 50 truncating information representative of physiological signal 130 based on saliency values 120 of at least some of the sample points 132 of information representative of physiological signal 130 and applying the truncated information representative of physiological signal to ML model 58 to generate an output, such as determining whether an ECAP is detected in the sensed physiological signal, may reduce the computational load and/or improve the performance of the ML model 58.

In some examples, processing circuitry 50 may be configured to adjust sensing, such as by a sensor that sensed physiological signal 130, sensing of a subsequent physiological signal based on the saliency values 120 of at least some of the sample points 132 of the physiological signal. For example, processing circuitry 50 may adjust a sensor to sense subsequent physiological signal at the sample points that have a saliency value above a saliency value threshold (e.g., a higher saliency value).

In some examples, ML model 58 is an example of a convolutional neural network (CNN) model, a deep learning model, or deep learning algorithm, trained to determine whether an ECAP signal from a supra-threshold control pulse is detected, determine a physiological parameter and/or health condition based on physiological data of a sensed physiological signal 130 or determine one or more particular features of the physiological signal 130. One or more of IMD 10, external device 12, server 94, and/or computing device(s) 100 may train, store, and/or utilize ML model 58, but other devices may apply inputs associated with a particular patient to ML model 58 in other examples. As discussed above, other types of machine learning and deep learning models or algorithms may be utilized in other examples. For examples, a convolutional neural network model of ResNet-18 may be used. Some non-limiting examples of models that may be used for transfer learning include AlexNet, VGGNet, GoogleNet, ResNet50, or DenseNet, etc. Some non-limiting examples of machine learning techniques include Support Vector Machines, K-Nearest Neighbor algorithm, and Multi-layer Perceptron. In some examples, ML model 58 may include a plurality of layers, such as a first layer (e.g., input layer), intermediate layers, and an output layer.

Although the techniques for applying truncated information representative of physiological signal to ML model 58 to generate an output is described as being performed by processing circuitry 50 of IMD 10, such techniques may be performed, in whole or part, by processing circuitry of any one or more devices of system 2, such as processing circuitry 80 of external device 12, processing circuitry 98 of server 94, or processing circuitry of one or more computing devices 100.

In some examples, stimulation circuitry 57 may be configured to generate electrical stimulation therapy and processing circuitry 50 may be configured to adjust one or more parameters that define the electrical stimulation therapy 57 based, at least, on the generated output by ML model 58.

In some examples, processing circuitry 50 may apply information representative of sensed physiological signal 130 to a first layer of ML model 58 to compute two linear transformations of the information representative of physiological signal to generate a single linear transformation of the information representative of physiological signal.

In some examples, data of information representative of physiological signal may comprise a particular bit size, such as 10-bits. In some examples, hardware that ML model 58 may be implanted on may be configured to receive data of different bit size, such as 8-bit or 16-bit. In some examples, processing circuitry 50 may be configured to discard 2 bits of the 10-bit data to provide an 8-bit input to apply to ML model 58.

In some examples, an input vector may represent a value of a sample point 132. In some examples, an input vector may be identified as X.
Input Vector→X

In some examples, ML model 58 may comprise a plurality of layers:
ML model is comprised of multiple layers l^{[n]}

In some examples, a first layer of ML model 58 may be represented by:
l^{[0]} [symbol] g(X·W^{[0]})

In some examples processing circuitry 50 may be configured to split up each data element having the particular bit size, such as 10-bit, into two data elements, a lower bits component and an upper bits component (input_low and input_high). In some examples, this may be done for every value in the input vector X to generate X′. For example,
X′=[X_{0:high}, X_{0:low}, X_{1:high}, X_{1:low}, . . . ]

In some examples, “input_low” may be equal to 7-bits and “input_high” may be equal to 3-bits.

In some examples, processing circuitry 50 may construct a weight matrix (e.g., a linter transformation) to reconstruct X. For example,

$${W}^{′} = [ \begin{matrix}{2}^{{n}_{low \_ bits}} & 0 & \\ 1 & 0 & \\ 0 & {2}^{{n}_{low \_ bits}} & … \\ 0 & 1 & \\ ⋮ & & ⋱\end{matrix} ]$$

In some examples, processing circuitry 50 may perform a linear transformation to transform the first layer of ML model 58. Such as
l^{[0]} [symbol] g((X′·W′)·W^{[0]})

In some examples, processing circuitry 50 may compute:
X′·W′

In some examples, the computational results of X′ and W′ may be stored in a wider bit width buffer within processing circuitry 50 for matrix multiplication with W[0] allowing higher resolution computations in the first layer.

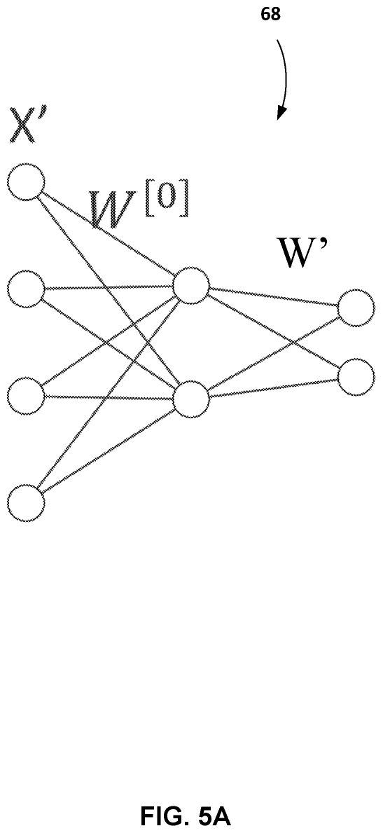

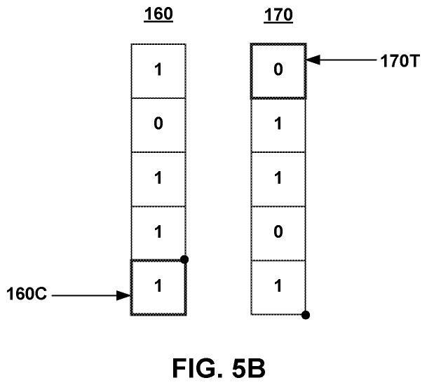

In some examples, as shown illustrated as an example in conceptual diagram FIG. 5A, ML model 58 may comprise a first layer 68 that includes a first linear transformation W′ and a second linear transformation W[0]. In some examples, processing circuitry 50 may apply the split data elements of the physiological signal, such as shown by input vector X′, to a first layer of the ML model 58 to compute two linear transformations W′ and W[0] of the physiological signal to generate an effective single linear transformation with higher resolution. In some examples, the generated single linear transformation may result with data elements having a size of 15-bits, which may be a better size for a data element to be applied to ML model 58 that is implemented on some particular hardware. In some examples, processing circuitry 50 may be configured to apply the linear transformation of the physiological signal to intermediate layer of ML mode 58 to generate an output of ML model 58.

In some examples, by applying two linear transformations in a first layer 68 of ML model 58 to generate a single linear transformation, processing circuitry 50 is able to change the data elements having a particular bit size, such as 10-bit, to a data element having a more effective bit size for particular hardware of an IMD, such as 15-bit. In some examples, processing circuitry 50 may be configured to compute two linear transformations of the physiological signal without using a non-linear activation function to generate the single linear transformation of the physiological signal.

FIG. 5B illustrates a conceptual diagram of truncating sensed physiological data by truncating at least one of the bits of an input vector at a particular position in the input vector and truncating at least one of the weights of a corresponding weight set of the ML model 58 at a position that is in an opposite direction than the position of the truncated at least one of the bits. In some examples, the least significant bit(s) are truncated from the input values and the most significant bit(s) are truncated from the weight values. In other examples, the most significant bit(s) are truncated from the input values and the least significant bit(s) are truncated from the weight values. In other examples, some of the most significant bit(s) and some of the least significant bits(s) are truncated from the input values and corresponding opposite least significant bit(s) and most significant bit(s) are truncated from the weight values. In some examples, an input vector may represent a value of a sample point 132.

In some examples, an input vector 160 of a sensed physiological signal 130 may have a particular size, such as 10-bits. In some examples, a ML model 58 may be configured to more effectively apply input vectors of a different size than of input vector 160, such as an input vector having a size of 8-bits. In some examples, as shown in FIG. 5B, processing circuitry 50 may be configured to truncate at least one-bit 160C of input vector 160. In some examples, processing circuitry 50 may be configured to truncate 2-bits of input vector 160, such as when input vector 160 includes 10-bits.

In some examples, processing circuitry 50 may be configured to truncate at least one of the weights 170T of the corresponding weight set 170 of ML model 58 at a position that is in an opposite direction than the position of the truncated bit 160C. For example, processing circuitry 50 may bit shift weight set 170 in an opposite direction of the bit 160C truncated of the input vector 160. In some examples, by truncating at least one of the weights 170T of the corresponding weight set 170 of ML model 58 at a position that is in an opposite direction than the position of the truncated bit 160C may enable processing circuitry 50 to dynamically shift the weight representation being applied to the truncated input vector.

In some examples, the number of bits and direction of truncation may be determined by the input values. For example, the upper (most significant) bits of the input values. In some examples, processing circuitry may default to truncating the most significant bits from the input values if the most significant bits are 0. In some examples, by truncating these particular values, you may lose no information from the input values. In some examples, to account for this truncation, processing circuitry may truncate the least significant bits from the weight values. As the upper bits of the input values fill with ones, processing circuitry 50 may preserve the most significant bits of the input values and sacrifice the least significant values, truncating the top N input bits of the input values that are all 0 and truncating the remaining bits needed to truncate from the lower bits in order to fit into a respective computational hardware. In some examples, equal, yet opposite, truncation may happen on the weight values. For example, if the most significant bit in the input vector is one, processing circuitry 50 may truncate the lower bits of the input values and upper bits of the weight values.

FIG. 6 is a block diagram illustrating an example configuration of components of external device 12. In the example of FIG. 6, external device 12 includes processing circuitry 80, communication circuitry 82, storage device 84, and user interface 86.

Processing circuitry 80 may include one or more processors that are configured to implement functionality and/or process instructions for execution within external device 12. For example, processing circuitry 80 may be capable of processing instructions stored in storage device 84. Processing circuitry 80 may include, for example, microprocessors, DSPs, ASICs, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80.

Communication circuitry 82 may include any suitable hardware, firmware, software or any combination thereof for communicating with another device, such as IMD 10. Under the control of processing circuitry 80, communication circuitry 82 may receive downlink telemetry from, as well as send uplink telemetry to, IMD 10, or another device. Communication circuitry 82 may be configured to transmit or receive signals via inductive coupling, electromagnetic coupling, Near Field Communication (NFC), Radio Frequency (RF) communication, Bluetooth, WiFi, or other proprietary or non-proprietary wireless communication schemes. Communication circuitry 82 may also be configured to communicate with devices other than IMD 10 via any of a variety of forms of wired and/or wireless communication and/or network protocols.

Storage device 84 may be configured to store information within external device 12 during operation. Storage device 84 may include a computer-readable storage medium or computer-readable storage device. In some examples, storage device 84 includes one or more of a short-term memory or a long-term memory. Storage device 84 may include, for example, RAM, DRAM, SRAM, magnetic discs, optical discs, flash memories, or forms of EPROM or EEPROM. In some examples, storage device 84 is used to store data indicative of instructions for execution by processing circuitry 80. Storage device 84 may be used by software or applications running on external device 12 to temporarily store information during program execution.

Data exchanged between external device 12 and IMD 10 may include operational parameters. External device 12 may transmit data including computer readable instructions which, when implemented by IMD 10, may control IMD 10 to change one or more operational parameters and/or export collected data. For example, processing circuitry 80 may transmit an instruction to IMD 10 which requests IMD 10 to export collected data (e.g., digitized physiological signals) to external device 12. In turn, external device 12 may receive the collected data from IMD 10 and store the collected data in storage device 84. In some examples, storage device 84 may store a ML model. Processing circuitry 80 may implement any of the techniques described herein to apply information representative of sensed physiological signal 130 to a ML model 58 to generate an output, such as whether an ECAP signal is detected, with respect to the sensed physiological signal 130.

A user, such as a clinician or patient 4, may interact with external device 12 through user interface 86. User interface 86 includes a display (not shown), such as a liquid crystal display (LCD) or a light emitting diode (LED) display or other type of screen, with which processing circuitry 80 may present information related to IMD 10, e.g., cardiac EGMs. In addition, user interface 86 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external device 12 and provide input. In other examples, user interface 86 also includes audio circuitry for providing audible notifications, instructions or other sounds to the user, receiving voice commands from the user, or both.

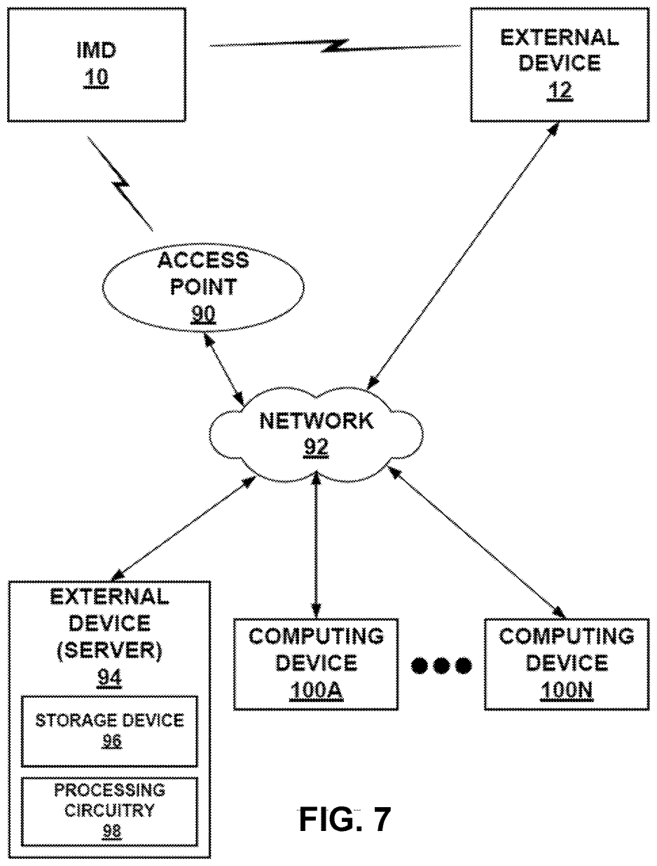

FIG. 7 is a block diagram illustrating an example system that includes an access point 90, a network 92, external computing devices, such as a server 94, and one or more other computing devices 100A-100N (collectively, “computing devices 100”), which may be coupled to IMD 10 and external device 12 via network 92, in accordance with one or more techniques described herein. In this example, IMD 10 may use communication circuitry 54 to communicate with external device 12 via a first wireless connection, and to communicate with an access point 90 via a second wireless connection. In the example of FIG. 7, access point 90, external device 12, server 94, and computing devices 100 are interconnected and may communicate with each other through network 92.

Access point 90 may include a device that connects to network 92 via any of a variety of connections, such as telephone dial-up, digital subscriber line (DSL), or cable modem connections. In other examples, access point 90 may be coupled to network 92 through different forms of connections, including wired or wireless connections. In some examples, access point 90 may be a user device, such as a tablet or smartphone, that may be co-located with the patient. IMD 10 may be configured to transmit data, such as physiological data, to access point 90. Access point 90 may then communicate the retrieved data to server 94 via network 92.

In some cases, server 94 may be configured to provide a secure storage site for data that has been collected from IMD 10 and/or external device 12. In some cases, server 94 may assemble data in web pages or other documents for viewing by trained professionals, such as clinicians, via computing devices 100. One or more aspects of the illustrated system of FIG. 7 may be implemented with general network technology and functionality, which may be similar to that provided by the Medtronic CareLink® Network.

In some examples, one or more of computing devices 100 may be a tablet or other smart device located with a clinician, by which the clinician may program, receive alerts from, and/or interrogate IMD 10. For example, the clinician may access data collected by IMD 10 through a computing device 100, such as when patient 4 is in in between clinician visits, to check on a status of a medical condition. In some examples, the clinician may enter instructions for a medical intervention for patient 4 into an application executed by computing device 100, such as based on a status of a patient condition determined by IMD 10, external device 12, server 94. or any combination thereof, or based on other patient data known to the clinician. Device 100 then may transmit the instructions for medical intervention to another of computing devices 100 located with patient 4 or a caregiver of patient 4. For example, such instructions for medical intervention may include an instruction to change a drug dosage, timing, or selection, to schedule a visit with the clinician, or to seek medical attention. In further examples, a computing device 100 may generate an alert to patient 4 based on a status of a medical condition of patient 4, which may enable patient 4 proactively to seek medical attention prior to receiving instructions for a medical intervention. In this manner, patient 4 may be empowered to take action, as needed, to address his or her medical status, which may help improve clinical outcomes for patient 4.

In the example illustrated by FIG. 7, server 94 includes a storage device 96, e.g., to store data retrieved from IMD 10, and processing circuitry 98. Although not illustrated in FIG. 7 computing devices 100 may similarly include a storage device and processing circuitry. Processing circuitry 98 may include one or more processors that are configured to implement functionality and/or process instructions for execution within server 94. For example, processing circuitry 98 may be capable of processing instructions stored in storage device 96. Processing circuitry 98 may include, for example, microprocessors, DSPs, ASICs, FPGAs, or equivalent discrete or integrated logic circuitry, or a combination of any of the foregoing devices or circuitry. Accordingly, processing circuitry 98 may include any suitable structure, whether in hardware, software, firmware, or any combination thereof, to perform the functions ascribed herein to processing circuitry 98. Processing circuitry 98 of server 94 and/or the processing circuitry of computing devices 100 may implement any of the techniques described herein to apply information representative of sensed physiological signal 130 to a ML model 58 to generate an output, such as whether an ECAP signal is detected, with respect to the sensed physiological signal 130.

Storage device 96 may include a computer-readable storage medium or computer-readable storage device. In some examples, storage device 96 includes one or more of a short-term memory or a long-term memory. Storage device 96 may include, for example, RAM, DRAM, SRAM, magnetic discs, optical discs, flash memories, or forms of EPROM or EEPROM. In some examples, storage device 96 is used to store data indicative of instructions for execution by processing circuitry 98.

Although the techniques for applying information representative of sensed physiological signal 130 to a ML model 58 to generate an output with respect to the sensed physiological signal 130 is described as being performed by processing circuitry 50 of IMD 10, such techniques may be performed, in whole or part, by processing circuitry of any one or more devices of system 2, such as processing circuitry 80 of external device 12, processing circuitry 98 of server 94, or processing circuitry of one or more computing devices 100.

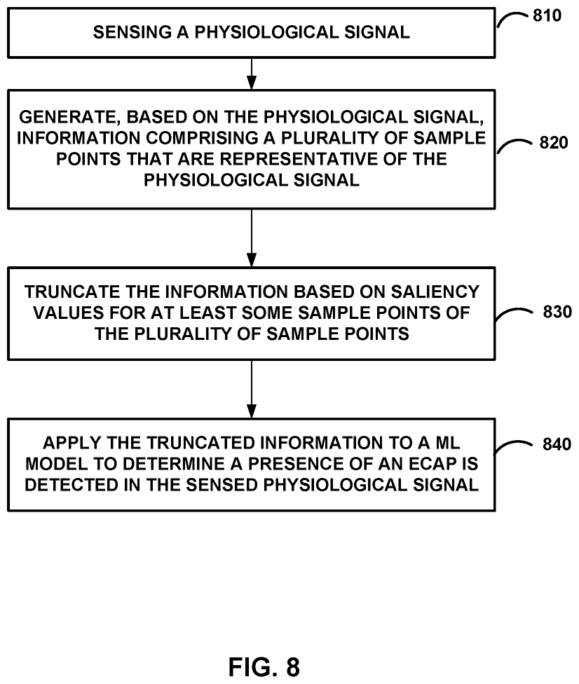

FIG. 8 is a flow diagram illustrating an example technique for a system as described herein. Processing circuitry 50 of IMD 10 will be described for exemplary purposes, but other devices and/or systems, alone or in combination, described herein may perform this technique. As shown in the example of FIG. 8, sensing circuitry 52 may be configured to sense a physiological signal 130 (810). In some examples, the physiological signal 130 may include and/or be based on signal(s) sensed by one or more sensors or electrodes of IMD 10.

In some examples, sensing circuitry 52 may be configured to generate, based on the physiological signal, information comprising a plurality of sample points that are representative of the physiological signal 130 (820).

Processing circuitry 50 may be configured to truncate the information based on saliency values 120 for at least some sample points of the plurality of sample points 132 (830). In some examples, processing circuitry 50 may compare the saliency values 120 of respective sample points 132 to a saliency value threshold and truncate the information based on the comparison. In some examples, processing circuitry 50 may truncate the information by discarding at least some of the sample points, such as artifact sample points 135 and/or late signal sample points 136, of the physiological signal 130 that have the lowest saliency values (e.g., below a saliency value threshold). In some examples, a saliency value threshold may be up to 0.005. In some examples, a saliency value threshold may be up to 0.01.

In some examples, ML model 58 may be trained on a plurality of training ECAP signal data and ML model 58 may determine and store saliency values 120 of at least some of the plurality sample points 132 of a physiological signal 130, such as an ECAP signal. In some examples, processing circuitry 50 may be configured to determine the saliency values 120 for the respective sample points 132. In some examples, processing circuitry 50 may be configured to truncate or average sample points of similar value to truncate the information.

Processing circuitry 50 may be configured to apply the truncated information to ML model 58 to determine a presence of an evoked compound action potential (ECAP) is detected in the sensed physiological signal 130 (840). In some examples, processing circuitry 50 may be configured to truncate the information and apply the truncated information to ML model 58 to generate an output, such as whether an ECAP signal from a supra-threshold control pulse is detected. In some examples, processing circuitry 50 may apply the truncated information to ML model 58 to generate an output, such as whether the detected a health condition status or prediction of the patient 4 or a determination of one or more features of the physiological signal 130 that may be indicative of a health condition status or prediction. In some examples, ML model 58 may also, or alternatively, determine a characteristic value of the ECAP present in the physiological signal. For example, the characteristic value may be indicative of a peak-to-peak value within the ECAP (e.g., N1 to P2 absolute value), area under one or more curves of the ECAP, number of ECAP peaks, latency of the ECAP signal, etc. In some examples, ML model 58 may output a value for subsequent stimulation pulses by incorporating ECAP characteristics and corresponding appropriate stimulation parameter values.

In some examples, processing circuitry 50 can truncate information based on saliency values 120 of at least some of the sample points 132 of physiological signal 130 and applying the truncated information to ML model 58 to generate an output, such as determining whether an ECAP is detected in the sensed physiological signal, may reduce the computational load and/or improve the performance of a ML model 58 positioned in IMD 10.

In some examples, processing circuitry 50 may be configured to generate a health event detection and/or prediction based, at least in part, on the output of ML model 58. In some examples, stimulation circuitry 57 may be configured to generate electrical stimulation therapy and processing circuitry 50 may be configured to adjust one or more parameters that define the electrical stimulation therapy 57 based, at least, on the generated output by ML model 58. For example, processing circuitry 50 may adjust an amplitude of subsequent stimulation pulses in response to a characteristic of the detected ECAP exceeding a threshold and/or deviating from a target value.

The following examples are illustrative of the techniques described herein.
Example 1: An implantable medical device includes sensing circuitry configured to: sense a physiological signal of a patient; and generate, based on the physiological signal, information comprising a plurality of sample points that are representative of the physiological signal; a memory configured to store a machine learning (ML) model; and processing circuitry configured to truncate the information based on saliency values for at least some sample points of the plurality of sample points; and apply the truncated information to the ML model to determine a presence of an evoked compound action potential (ECAP) in the sensed physiological signal.
Example 2: The implantable medical device of example 1, wherein the processing circuitry is further configured to: compare the saliency values to a saliency value threshold; and truncate the information based on the comparison.
Example 3: The implantable medical device of example 2, wherein the processing circuitry is further configured to truncate the information by discarding at least some sample points having a saliency value below the saliency value threshold.
Example 4: The implantable medical device of example 3, wherein the saliency value threshold is based on a pre-determined amount or percentage of plurality of sample points to be truncated.
Example 5: The implantable medical device of any of examples 2-4, wherein the processing circuitry is further configured to: adjust, based on the saliency values of respective sample points, weighting of the ML model to apply to the respective sample points of the truncated information.
Example 6: The implantable medical device of any of examples 2-5, wherein the processing circuitry is further configured to: control the sensing circuitry to adjust, based on the saliency values of at least some of the plurality of sample points, sensing of a subsequent physiological signal.
Example 7: The implantable medical device of any of examples 2-6, wherein the ML model is trained to determine the saliency values for the plurality of sample points of the information.
Example 8: The implantable medical device of any of examples 1-7, wherein the processing circuitry is further configured to: apply the information to a first layer of the ML model to compute two linear transformations of the information to generate a single linear transformation of the information e; and apply the single linear transformation of the information to intermediate layers of the ML model to generate an output.
Example 9: The implantable medical device of example 8, wherein the processing circuitry is configured to compute the two linear transformations of the information by at least computing the two linear transformations of the information without a non-linear activation function to generate the single linear transformation of the information.
Example 10: The implantable medical device of any of examples 1-7, wherein portions of the information are represented by a plurality of input vectors having a particular size, wherein each bit of an input vector of the plurality of input vectors has a corresponding weight of a corresponding weight set of the ML model, and wherein, for each of the plurality of input vectors, the processing circuitry is further configured to: truncate at least one of the bits of the input vector at a particular position in the input vector; and truncate at least one of the weights of the corresponding weight set at a position that is in an opposite direction than the position of the truncated at least one bits.
Example 11: The implantable medical device of example 10, wherein the size of the input vector is 10 bits.
Example 12: The implantable medical device of any of examples 1 through 11, further includes determine a characteristic of the ECAP; and control, based on the characteristic of the ECAP, the stimulation circuitry to deliver electrical stimulation to a patient.
Example 13: A method includes sensing, by sensing circuitry, a physiological signal of a patient; generating, by the sensing circuitry and based on the physiological signal, information comprising a plurality of sample points that are representative of the physiological signal; truncating, by processing circuitry, the information based on saliency values of at least some sample points of the plurality of sample points; and applying, by the processing circuitry, the truncated information to a machine learning (ML) model to determine a presence of an evoked compound action potential (ECAP) is detected in the physiological signal.
Example 14: The method of example 13, further includes comparing, by the processing circuitry, the saliency values to a saliency value threshold; and truncating, by the processing circuitry, the information based on the comparison.
Example 15: The method of example 14, further includes truncating, by the processing circuitry, the information by discarding the at least some sample points having a saliency value below the saliency value threshold.
Example 16: The method of any of examples 13-15, further includes adjusting, by the processing circuitry and based on the saliency values of respective sample points, weights of the ML model; and applying, by the processing circuitry, the adjusted weights of the ML model to the respective sample points of the truncated information.
Example 17: The method of any of examples 13-16, further includes adjusting, based on the saliency values of the at least some of the plurality of sample points, sensing, by the sensing circuitry, of a subsequent physiological signal.
Example 18: The method of any of examples 13-17, wherein the ML model is trained to determine the saliency values for the plurality of sample points of the information.
Example 19: The method of any of examples 13-18, further includes applying, by the processing circuitry, the information to a first layer of the ML model to compute two linear transformations of the information to generate a single linear transformation of the information; and applying, by the processing circuitry, the single linear transformation of the information to intermediate layers of the ML model to generate an output.
Example 20: The method of any of examples 13-18, wherein portions of the information are represented by a plurality of input vectors having a particular size, wherein each bit of an input vector of the plurality of input vectors has a corresponding weight of a corresponding weight set of the ML model, wherein, the method further comprises: for at least one input vector of the plurality of input vectors: truncating, by the processing circuitry, at least one of the bits of the input vector at a particular position in the input vector; and truncating, by the processing circuitry, at least one of the weights of the corresponding weight set at a position that is in an opposite direction than the position of the truncated at least one bits.
Example 21: A computer-readable medium comprising instructions that, when executed, causes processing circuitry of a medical system to perform the method of any of examples 13-20.

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

Various examples have been described. These and other examples are within the scope of the following claims.

What is claimed is:

1. An implantable medical device comprising:
sensing circuitry configured to:
sense a physiological signal of a patient; and
generate, based on the physiological signal, information comprising a plurality of sample points that are representative of the physiological signal;
a memory configured to store a machine learning (ML) model; and
processing circuitry configured to:
truncate the information based on saliency values for at least some sample points of the plurality of sample points; and
apply the truncated information to the ML model to determine a presence of an evoked compound action potential (ECAP) in the sensed physiological signal.

2. The implantable medical device of claim 1, wherein the processing circuitry is further configured to:
compare the saliency values to a saliency value threshold; and
truncate the information based on the comparison.

3. The implantable medical device of claim 2, wherein the processing circuitry is further configured to truncate the information by discarding at least some sample points having a saliency value below the saliency value threshold.

4. The implantable medical device of claim 3, wherein the saliency value threshold is based on a pre-determined amount or percentage of plurality of sample points to be truncated.

5. The implantable medical device of claim 2, wherein the processing circuitry is further configured to:
adjust, based on the saliency values of respective sample points, weighting of the ML model to apply to the respective sample points of the truncated information.

6. The implantable medical device of claim 2, wherein the processing circuitry is further configured to:
control the sensing circuitry to adjust, based on the saliency values of the at least some of the plurality of sample points, sensing of a subsequent physiological signal.

7. The implantable medical device of claim 2, wherein the ML model is trained to determine the saliency values for the plurality of sample points of the information.

8. The implantable medical device of claim 1, wherein the processing circuitry is further configured to:
apply the information to a first layer of the ML model to compute two linear transformations of the information to generate a single linear transformation of the information e; and
apply the single linear transformation of the information to intermediate layers of the ML model to generate an output.

9. The implantable medical device of claim 8, wherein the processing circuitry is configured to compute the two linear transformations of the information by at least computing the two linear transformations of the information without a non-linear activation function to generate the single linear transformation of the information.

10. The implantable medical device of claim 1, wherein portions of the information are represented by a plurality of input vectors having a particular size,
wherein each bit of an input vector of the plurality of input vectors has a corresponding weight of a corresponding weight set of the ML model, and
wherein, for each of the plurality of input vectors, the processing circuitry is further configured to:
truncate at least one of the bits of the input vector at a particular position in the input vector; and
truncate at least one of the weights of the corresponding weight set at a position that is in an opposite direction than the position of the truncated at least one bits.

11. The implantable medical device of claim 10, wherein the size of the input vector is 10 bits.

12. The implantable medical device of claim 1, further comprising stimulation circuitry, and wherein the processing circuitry is configured to:
determine a characteristic of the ECAP; and
control, based on the characteristic of the ECAP, the stimulation circuitry to deliver electrical stimulation to a patient.

13. A method comprising:
sensing, by sensing circuitry, a physiological signal of a patient;
generating, by the sensing circuitry and based on the physiological signal, information comprising a plurality of sample points that are representative of the physiological signal;
truncating, by processing circuitry, the information based on saliency values of at least some sample points of the plurality of sample points; and
applying, by the processing circuitry, the truncated information to a machine learning (ML) model to determine a presence of an evoked compound action potential (ECAP) is detected in the physiological signal.

14. The method of claim 13, further comprising:
comparing, by the processing circuitry, the saliency values to a saliency value threshold; and
truncating, by the processing circuitry, the information based on the comparison.

15. The method of claim 14, further comprising:
truncating, by the processing circuitry, the information by discarding the at least some sample points having a saliency value below the saliency value threshold.

16. The method of claim 13, further comprising:
adjusting, by the processing circuitry and based on the saliency values of respective sample points, weights of the ML model; and
applying, by the processing circuitry, the adjusted weights of the ML model to the respective sample points of the truncated information.

17. The method of claim 13, further comprising:
adjusting, based on the saliency values of at least some of the plurality of sample points, sensing, by the sensing circuitry, of a subsequent physiological signal.

18. The method of claim 13, further comprising:
applying, by the processing circuitry, the information to a first layer of the ML model to compute two linear transformations of the information to generate a single linear transformation of the information; and
applying, by the processing circuitry, the single linear transformation of the information to intermediate layers of the ML model to generate an output.

19. The method of claim 13, wherein portions of the information are represented by a plurality of input vectors having a particular size,
wherein each bit of an input vector of the plurality of input vectors has a corresponding weight of a corresponding weight set of the ML model,
wherein, the method further comprises:
for at least one input vector of the plurality of input vectors:
truncating, by the processing circuitry, at least one of the bits of the input vector at a particular position in the input vector; and
truncating, by the processing circuitry, at least one of the weights of the corresponding weight set at a position that is in an opposite direction than the position of the truncated at least one bits.

20. A computer-readable medium comprising instructions that, when executed, causes processing circuitry of a medical system to:
sense a physiological signal of a patient;
generate, based on the physiological signal, information comprising a plurality of sample points that are representative of the physiological signal;
truncate the information based on saliency values of at least some sample points of the plurality of sample points; and
apply the truncated information to a machine learning (ML) model to determine a presence of an evoked compound action potential (ECAP) is detected in the physiological signal.
